(19) United States
(12) Patent Application Publication
(10) Pub. No.: US 2005/0085708 A1
(43) Pub. Date: Apr. 21, 2005

(54) System and method for preparation of cells for 3D image acquisition

(75) Inventors: Mark E. Fauver, Seattle, WA (US); Alan C. Nelson, Gig Harbor, WA (US); John Richard Rahn, Sammamish, WA (US); Eric J. Seibel, Seattle, WA (US); Florence W. Patten, Issaquah, WA (US); Shawn McGuire, Seattle, WA (US)

Correspondence Address: GEORGE A LEONE, SR, 2150 128TH AVENUE, NW, MINNEAPOLIS, MN, 55448, US

(73) Assignees: University of Washington; VisionGate, Inc.

(21) Appl. No.: 10/968,645

(22) Filed: Oct. 19, 2004

Related U.S. Application Data
(63) Continuation-in-part of application No. 10/126,026, filed on Apr. 19, 2002.

(51) Int. Cl. A61B 5/05

(52) U.S. Cl. 600/407

(57) ABSTRACT

The present invention provides a method for embedding particles in a solid structure including the steps of extruding a slurry of particles and a polymeric solution into a linear polymer medium having particles embedded into a polymer portion; and curing the polymer portion of the linear polymer medium.

### RELATED APPLICATIONS

This application claims the benefit of the priority date and is a continuation-in-part of co-pending U.S. patent application Ser. No. 10/126,026, filed Apr. 19, 2002, of Nelson entitled “VARIABLE-MOTION OPTICAL TOMOGRAPHY OF SPECIMEN PARTICLES,” the disclosure of which is incorporated herein by this reference.

This application is also related to concurrently filed application to Fauver et al. entitled, “IMPROVEMENTS IN OPTICAL PROJECTION TOMOGRAPHY MICROSCOPE,” attorney docket no. 60097US that is assigned to the same assignees as the present application and the disclosure of which is hereby incorporated by reference.

### FIELD OF THE INVENTION

The present invention relates generally to the field of specimen preparations and, more particularly, to a method for preparing cells in transport meddium such as a thixotropic gel or a polymer medium for use in three dimensional image acquisition.

### BACKGROUND OF THE INVENTION

For some imaging applications, it is desirable to generate optical information in three dimensions from a thick specimen. Three-dimensional optical information can be generated using the techniques of computed tomographic image reconstruction, in which successive projection images are acquired from a number of perspectives. The perspectives usually form an arc of substantially 180 degrees about the specimen. For three-dimensional imaging, it is important that each perspective receive light in approximately the same manner, without large alterations in the transmitted light due to the optical characteristics or dimensions of the sample container. For this reason, methods such as placing the samples on a flat surface, such as a microscope slide, are not suitable, as the optical thickness of the slide and of the cover-glass (if one is used) will vary significantly as the slide is rotated by 180 degrees about one of its lateral dimensions.

One example of embedding specimens within a standard flat microscope slide format has been published by Reymond and Pickett-Heaps (1983), entitled “A Routine Flat Embedding Method for Electron Microscopy of Microorganisms Allowing Selection and Precisely Orientated Sectioning of Single Cells by Light Microscopy,” Journal of Microscopy, Vol. 130, Pt. 1, April 1983, pp.79-84. Reymond and Pickett-Heaps describe a molding technique for making thin slides of embedding material containing cells for optical sample preparation for electron microscopy. Unfortunately, variations from multiple perspectives when viewing a slide can produce large optical aberrations, as well as a large degree of scattering and absorption. Such large optical aberrations may render the projections taken unusable, especially if taken from a perspective close to the plane of the slide.

A more effective type of sample container should have approximately equivalent optical thickness about an arc of 180 degrees. Geometries that may meet this requirement include hollow tubes having concentric inner and outer walls, or tubes with concentric polygonal inner and outer walls Examples of a sample chamber design for optical applications are shown in Schrader, “Sample Arrangement for Spectrometry, Method for the Measurement of Luminescence and Scattering and Application of the Sample Arrangement,” U.S. Pat. No. 4,714,345, issued Dec. 22, 1987; and Gilby, “Laser Induced Fluorescence Capillary Interface,” U.S. Pat. No. 6,239,871, issued May 25, 2001.

When a specimen comprises individual biological cells, or other material with spatial dimensions of roughly 100 microns or less, there may be additional requirements for the chamber. Because of the small sizes involved, it may prove difficult to insert the cells into, for example, a small capillary tube. Glass capillaries tend to be brittle, and hence easily broken. If the sample to be examined includes a large number of cells, strung out along a long length of glass capillary tubing, then their storage and transport can be very difficult. The alternative method of using a large number of short tubing segments is equally unappealing. Further, if the mechanism for insertion makes use of capillary rise, then the method may be subject to constraints imposed by the chemistry related to the capillary rise. This can be a particular problem when the cell preparation and presentation medium have specific requirements of their own, which may be incompatible with the requirements of the glass-solvent interfacial chemistry.

One drawback of immobilizing the cells within a tube, using such means as injecting epoxies or other optical adhesives into the tube, often results in empty spaces within the tube due to volume change upon curing or upon evaporation of the epoxy's solvent. Further, curing may not be possible due to the enclosed, unventilated volume within the tube. Thus the cells may not be fully immobilized, and the presence of empty spaces, such as bubbles, may contribute to spurious scattering effects during image acquisition. Yet another issue arises due to the possible mismatch between the refractive indices of the sample container, the medium within which the cells are suspended, and the cells themselves. A mismatch between the first two can result in undesirable lensing effects and aberrations of the light rays. At the same time, for some biomedical applications it may be desirable to examine the cell nuclei, while excluding the cell cytoplasms from consideration. Thus, in using a glass tube with a suspending medium, it may become necessary to match the refractive indices of three materials, namely, the tube walls, the suspending medium, and the cell cytoplasm. An example of refractive-index matching is described by Albert et al., in “Suspended Particle Displays and Materials for Making the Same,” U.S. Pat. No. 6,515,649, issued Feb. 4, 2003.

Another issue arises when a chain of custody is required, as may be the case in a biomedical screening application. See, for example, the article by Nicewarner-Peña et al., entitled “Submicrometer Metallic Barcodes,” Science 294, 137 (2001).

In contrast to conventional methods and to overcome the problems noted hereinabove, one method of the present invention uses polymeric materials that are less brittle than glass, and thus easier to handle. Polymeric materials can be made flexible, allowing a single length to be wrapped into a compact roll for convenient handling and storage. Further in contrast to conventional methods, the method of the present invention does not require entrapment of polymers inside a small volume, and permits a uniform, homogeneous medium in which cells are presented. By using the same material as both the sample container and as the suspending medium, the method of the present invention reduces the problem to matching the polymer's refractive index with that of the cytoplasm. If it is desirable to also image the cytoplasm, then refractive-index matching is not required. In the present invention, chemical interactions between the sample and its container play a less significant role.

### SUMMARY OF THE INVENTION

The present invention provides a method for embedding particles in a solid structure including the steps of extruding a slurry of particles and a polymeric solution into a linear polymer medium having particles embedded into a polymer portion; and curing the polymer portion of the linear polymer medium.

### BRIEF DESCRIPTION OF THE DRAWINGS

FIG. 1 illustrates an extrusion method of embedding a specimen in a solid medium, as contemplated by one embodiment of the present invention.

FIG. 2 illustrates an alternate extrusion method of embedding a specimen in a solid medium, as contemplated by another embodiment of the present invention using a vertical orientation and vibration to create microdroplets, each microdroplet containing a single cell.

FIG. 3 schematically shows a functional block diagram of an example of a system and method for embedding a specimen in a solid medium using pressurized slurry, as contemplated by one embodiment of the present invention.

FIG. 4 shows an example of an optical tomography system employing multiple sets of source-detector pairs along a series of different specimens where the specimens are prepared as contemplated by an embodiment of the invention.

FIG. 5 shows schematically an example illustration of cells embedded into a linear polymer medium for use in variable motion optical tomography as contemplated by an embodiment of the present invention.

FIG. 6 and FIG. 6A schematically illustrate a front view and end view respectively of a system for using hydrodynamic focusing for centering cells in a cylindrically shaped medium.

FIG. 7 schematically illustrates a side view of the system for using hydrodynamic focusing for centering cells in cylindrically-shaped medium as shown in FIG. 6.

While the novel features of the invention are set forth with particularity in the appended claims, the invention, both as to organization and content, will be better understood and appreciated, along with other objects and features thereof, from the following detailed description taken in conjunction with the drawings.

### DETAILED DESCRIPTION OF THE PREFERRED EMBODIMENTS

The method and apparatus of the invention is here described with reference to specific examples that are intended to be illustrative and not limiting. Generally, a specimen to be examined is embedded, or encapsulated, in a homogeneous, optically clear medium, such as a polymer. The suspension comprising the specimen and the medium can be shaped to provide a desired geometry. Upon making the medium into a solid, either by curing or by evaporating the solvent, a flexible, optically clear solid suspension is formed. The solid suspension can be used as a means for supporting, presenting, handling, and storing the specimen. The method and apparatus of the invention is amenable to additional features such as matching of the refractive indices of the materials in the solid suspension and the inclusion of microscopic barcodes to facilitate identification of the specimen. The components used can be made as inexpensive, disposable items, as is necessary when the specimens are biomedical samples.

The medium may be formed by extrusion and subsequent curing of a slurry composed of cells and polymers in solution; by micromolding and subsequent curing of a such a slurry; or by forcing such a slurry into a microcapillary tube, followed by curing. The method disclosed may be useful in applications requring high throughput of cells as part of a three-dimensional imaging system. The manufacturing method can be extended by forming distinct droplets of unpolymerized polymer to form individual spheres encapsulating an individual cell.

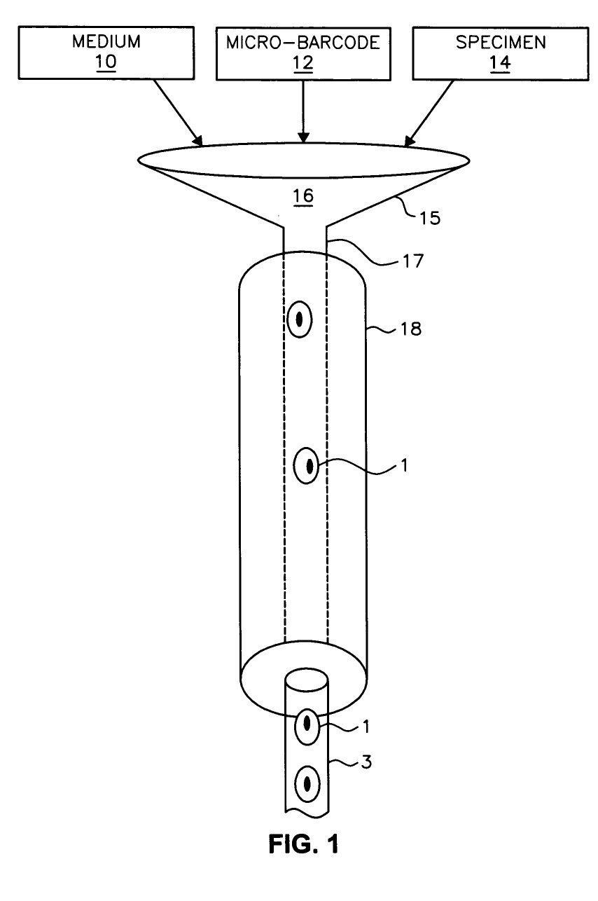

Referring now to FIG. 1, there illustrated is an extrusion method of embedding a specimen in a solid medium, as contemplated by one embodiment of the present invention. There shown is a slurry of particles 16 including a mixture of a mounting medium 10 and a specimen 14. The mounting medium 10 may advantageously be a polymeric solution or equivalent. In one useful application the specimen 14 comprises a biological specimen, including particles, as for example, at least one cell, biological cells harvested for cancer diagnosis, a cell nucleus, a nucleus, an embedded molecular probe and/or the like. Optionally, a micro-barcode source 12 may insert a micro-barcode 44 into the slurry 16.

The slurry may be in a container 15 that is coupled to an injection device 17, wherein the container 15 may advantageously be a disposable container and the injection device 17 is a conventional injection molding device or equivalents. A linear polymer medium 3, comprising particles 1 emerges from the molding tube 18 and is cured by heat curing or ultra-violet absorption into a solid cylinder of polymer having embedded particles. In one embodiment of the apparatus of the invention, the injection device 17 operates to regulate the spacing between each object along the length of the linear polymer medium 3. The polymeric solution preferably comprises a polymer selected to be substantially transparent to visible light and provide, upon solidification and curing, a matching of its index of refraction with the index of refraction of a portion of the particles contained in the slurry 16.

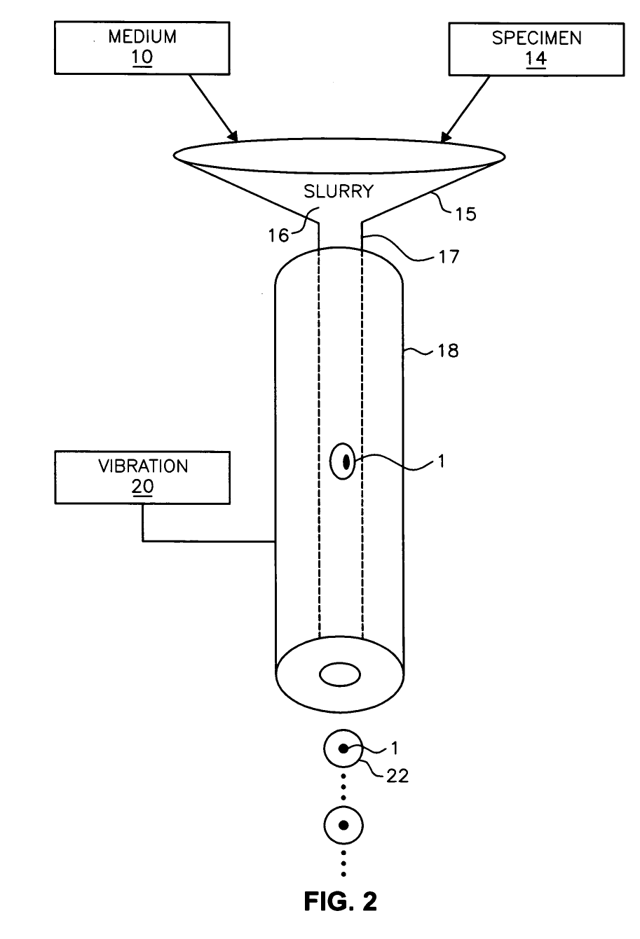

Referring now to FIG. 2, there illustrated is an alternate extrusion method of embedding a specimen particle in a solid medium, as contemplated by another embodiment of the present invention using a vertical orientation and vibration to create microdroplets, each containing a single particle 1, such as a biological cell, especially a human cell. The apparatus is constructed substantially identically as the apparatus described hereinabove with reference to FIG. 1, with the addition of a vibration device 20. The vibration device 20 may advantageously comprise a conventional vibration element such as a piezoelectric element or equivalent device. The vibration device 20 is adjusted to produce individual microspheres 22 of hardened polymer.

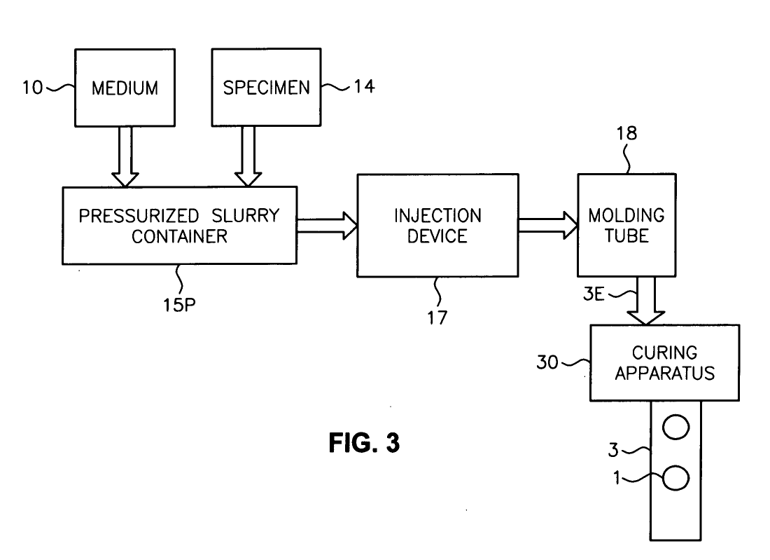

Referring now to FIG. 3, a functional block diagram of an example of a system and method for embedding a specimen in a solid medium using a pressurized slurry, as contemplated by one embodiment of the present invention is schematically shown. The system includes a slurry of specimen 14 and mounting medium 10 in a pressurized slurry container 15P. The pressurized slurry container 15P is coupled to an injection device 17 coupled to a molding tube 18, such as a microcapillary tube, and an extruded linear polymer medium 3E is solidified in curing apparatus 30, resulting in a solidified linear polymer medium 3 having embedded particles 1.

An alternative method for embedding particles in a solid structure includes micromolding a slurry including particles and a polymeric solution; and curing the polymer portion of the slurry to form a solid specimen carrier. The step of micromolding may advantageously include using a disposable mold. The step of micromolding may advantageously also include an intermediate step of using an injection device to regulate the spacing between each object along the length of solid specimen carrier. Other combinations of steps and elements may be carried out as described above.

Another alternative method in accordance with the principles of the present invention for embedding particles in a solid structure, includes the steps of pressurizing a slurry including particles and a polymeric solution to force the slurry into a microcapillary tube, and curing the polymer portion of the slurry to form a solid specimen carrier. Other combinations of steps and elements may be carried out as described above.

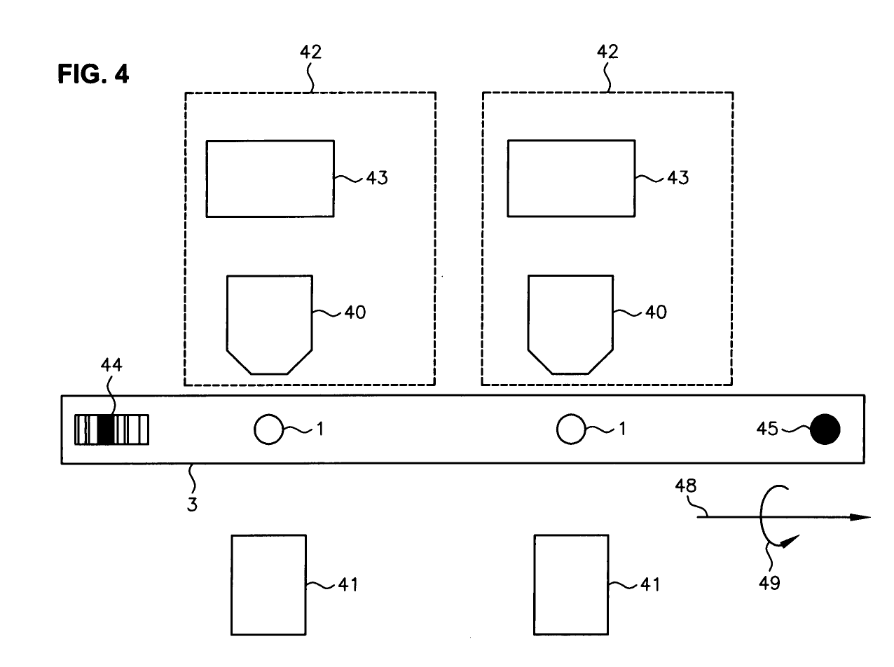

Referring now particularly to FIG. 4, an example of an optical tomography system employing multiple sets of source-detector pairs along a series of different specimens, the specimens being embedded in a rigid medium as contemplated by an embodiment of the invention, is schematically illustrated. A plurality of specimens such as cells 1 or nuclei 2 may be carried by a rigid medium having one or more fiducials 45 for registration. Each of the multiple sets of pseudo-projection viewing subsystems include an image detector 42 such as a CCD or CMOS camera, disposed to receive image information from an objective lens 40, illuminated by an illumination system 41 comprised of a illumination source, condenser lens, and two apertures. The rigid medium may comprise an extruded linear polymer medium 3 or other equivalent medium. Specimen samples are moved through various stations of source-detector pairs along the direction indicated by arrow 48. Each fiducial 45, such as an opaque microsphere, aids in detecting specimen positioning and positional shifts during translation and/or rotation, and may be used with conventional automatic image registration techniques on the images being integrated on the image detector, or on individual images that are being summed for a single integration by the computer. The registration of the multiple projections is corrected as the rigid medium is rotated as indicated by arrow 49. In contrast to prior art techniques, the present invention moves the objective lens with respect to the specimen to scan the focal plane continuously and sums the images optically at the detector, and is not restricted to summing individual images acquired and summed only electronically. Unique indicia 44, such as a micro-barcode, may be placed to identify and to maintain a chain of custody for each of the plurality of specimens.

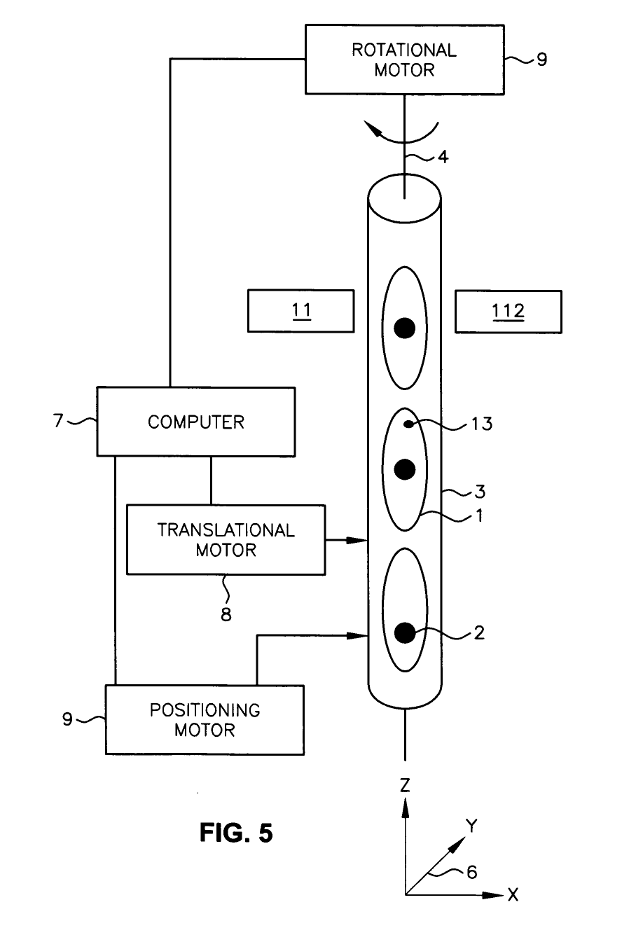

Referring now to FIG. 5, there shown schematically is an example illustration of cells embedded into a linear polymer medium as contemplated by an embodiment of the present invention. In this example embodiment, a section of the linear polymer medium 3 is filled with particles 1, such as cells, that are embedded rigidly into the linear polymer medium. Each of the cells may include a nucleus 2. The linear polymer medium 3 has a central axis 4 oriented with reference to a coordinate system 6 having coordinates in the x, y and z-directions. In some instances, at least one molecular probe 13 may be bound within the cell. A computer 7 is coupled to provide control signals to a rotational motor 5 and a translational motor 8. It will be recognized that equivalent arrangements of one or more motors, gears or fluidics or other means of generating motion may also be employed to achieve the necessary translational and rotational motion of the linear polymer medium or other substrate. In some cases, one or more of the motors may be replaced by manual positioning devices or gears or by other means of generating motion such as hydraulic or piezoelectric transducers. The axis of translation is the z-axis, and rotation is around the z-axis. The positioning motor 9 is coupled to move the cell in a plane defined by the x, y-axes, substantially perpendicular to the central axis for the purpose of centration, as necessary.

It will be recognized that the curved surface of the linear polymer medium will act as a cylindrical lens and that this focusing effect may not be desirable in a projection system. Those skilled in the art will appreciate that the bending of photons by the linear polymer medium can be eliminated if the spaces between (a) the illumination source 11 and the linear polymer medium and (b) between the linear polymer medium surface and the detector 112 are filled with a material whose index of refraction matches that of the linear polymer medium and that the linear polymer medium can be optically coupled (with oil or a gel, for example) to the space filling material. When index of refraction differences are necessary, for instance due to material choices, then at minimum the index of refraction difference should only exist between flat surfaces in the optical path. Illumination source 11 and detector 112 form a source-detector pair. Note that one or more source-detector pairs may be employed.

Consider the present example of cells embedded into a linear polymer medium. The cells may preferably be embedded single file so that they do not overlap. The density of embedding whole cells of about 100 microns in diameter into a linear polymer medium with diameter less than 100 microns can be roughly 100 cells per centimeter of linear polymer medium length. For bare nuclei of about 20 microns in diameter, the embedding can be roughly 500 nuclei per centimeter of linear polymer medium length where the linear polymer medium diameter is proportional to the object size, about 20 microns in this case. Thus, within several centimeters of linear polymer medium length, a few thousand non-overlapping bare nuclei can be embedded. By translating the linear polymer medium along its central axis 4, motion in the z-direction can be achieved. Moving the linear polymer medium in the x, y-directions allows objects within the linear polymer medium to be centered, as necessary, in the reconstruction cylinder of the optical tomography system. By rotating the linear polymer medium around its central axis 4, a multiplicity of radial projection views can be produced. Moving the linear polymer medium in the z-direction with constant velocity and no rotation simulates the special case of flow optical tomography.

One advantage of moving a linear polymer medium filled with cells that are otherwise stationary inside the linear polymer medium is that objects of interest can be stopped, then rotated, at speeds that permit nearly optimal exposure for optical tomography on a cell-by-cell basis. That is, the signal to noise ratio of the projection images can be improved to produce better images than may be usually produced at constant speeds and direction typical of flow systems. Objects that are not of interest can be moved out of the imaging system swiftly, so as to gain overall speed in analyzing cells of interest in a sample consisting of a multitude of cells. Additionally, the ability to stop on an object of interest, and then rotate as needed for multiple projections, nearly eliminates motion artifacts. Still further, the motion system can be guided using submicron movements and can advantageously be applied in a manner that allows sampling of the cell at a resolution finer than that afforded by the pixel size of the detector. More particularly, the Nyquist sampling criterion could be achieved by moving the system in increments that fill half a pixel width, for example. Similarly, the motion system can compensate for the imperfect fill factor of the detector, such as may be the case if a charge-coupled device with interline-transfer architecture is used.

Cell Preparation for Step Flow Actuation of Cells

An alternate method for cell preparation is described hereinbelow for step flow actuation of cells. Step flow actuation of cells requires that cells be embedded in a highly viscous, preferably thixotropic, liquid, for example, having a typical viscosity>1 million centipoises (cps). Unlike flow cytometry, where non-viscous fluids are used to transport cells, and the parabolic velocity profile is used for hydrodynamic focusing to center cells in the tube, step flow has a flat velocity profile. Because of the high viscosity of the carrier medium, cells remain stationary when the medium has zero velocity. Using this type of medium for transport, cells can be actuated into the field of view for measurement, but then stopped so that images of the cell can be acquired without blurring. Furthermore, the cell can be rotated around one axis in a stepwise manner for tomographic imaging purposes.

Herein is described a method for preparing cells and embedding them into a suitable high viscosity gelatinous medium, a method for actuation of the cells embedded in the high viscosity gelatinous medium, and the manner in which the method allows detailed high resolution imaging of the cell.

The method for preparation of cells for embedding in a high viscosity medium suitable for imaging involves transfer of cells into a suitable solvent which does not chemically react with the carrier medium, in this example the solvent is xylene, and centrifugation of the resulting cell/solvent mixture into an optical gel such as, for example, Nye OC431A. Nye OC431A optical gel advantageously has high viscosity so that cells remain stationary when desired, and a refractive index matched to the silica microcapillary tube that serves as the conduit for cell actuation. Refractive index matching both inside the tube, and outside the tube between two flat parallel surfaces is employed for high resolution imaging in order to minimize optical distortions. Since it is likely that the solvent is retained within the fixed stained cell after centrifugation into the optical gel, the solvent also may affect refractive index matching of the interior of the cell to the optical gel (or other carrier medium). Thus, the solvent used may preferably be selected to match the surface refractive index.

As noted above, a conventional flow cytometer uses a very low viscosity carrier medium, typically water having a dynamic viscosity=1 centipoise (cps). In contrast, a step flow system and method constructed in accordance with the present invention uses a moderate-to-high viscosity carrier medium. One objective of the step flow system is to ensure registration of multiple images taken sequentially on a specimen. In the case of optical tomography, for example, a sequence of images is acquired from multiple angles. Registration is important, especially for doing 3D tomographic reconstruction from such a data set. In order to keep acceptable registration, the viscosity of the carrier medium may be determined from the following relationship,

$$η = \frac{2 {R}^{2} ( {ρ}_{specimen} - {ρ}_{medium} ) a}{9 {v}_{sed}}$$

where η is dynamic viscosity of the carrier medium,
R is the radius of the cell,
ρ is the density of the specimen and the medium as noted,
a is the acceleration, and
v_{sed} is the sedimentation velocity.

In order to prevent loss of registration between multiple images, the specimen cannot move more than a specified distance d over the period of time it takes to acquire all images. The maximum acceptable distance d can be defined to be 0.25 of the desired image resolution. In one example, the maximum acceptable distance d equals 0.25(0.5 microns)=0.125 microns. Time T for acquisition of a data set comprising 250 images typically ranges from 250 msec to 60 sec. Thus the maximum sedimentation velocity
v_{sed=d/T}
   such that
0.2×10⁻⁶ cm/sec≦v_{sed}≦0.5×10⁻⁴ cm/sec.
   If the specimen were a single cell nucleus, let R=5 microns=5×10⁻⁴ cm and ρ_{specimen}=1.4 g/cm³
(and for a preferred optical gel medium ρ_{medium}=1.06 g/cm³)

$$η = \frac{2 {R}^{2} ( {ρ}_{specimen} - {ρ}_{medium} ) g}{9 ( d \text{/} T )}$$

Inserting these values, the dynamic viscosity η of a useful medium is >37 centipoise (cps) for T=250 msec. For a time interval T=60 sec, η is >8800 cps. The density of the medium itself may also be altered to yield an acceptably low sedimentation rate over the time period T. However, in considering acceleration and deceleration of the carrier medium, it is advantageous to have the density of the specimen similar to the density of the carrier medium so that movement of the specimen relative to the carrier medium is minimized.

Higher viscosities may be useful, though higher viscosities limit the throughput rate of specimen processed by the instrument, as well as limiting the acceleration and deceleration of the carrier medium during actuation. If other external forces, such as that due to centripetal acceleration caused by spinning the microcapillary tube around its axis, are present, the viscosity of the carrier medium may be increased to keep specimen positional stability to an acceptable level.

In the case of a step flow system using a moderate-to-high viscosity carrier medium, hydrodynamic focusing is unnecessary for particle positional stability over the total measurement time T. Hydrodynamic focusing may be employed to improve centration of the cell specimen with the microcapillary tube axis, but is not critical for positional stability. In the case where the carrier medium exhibits non-Newtonian behavior, a flattened velocity profile may occur, in which case it becomes even more necessary to employ increased carrier medium viscosity for specimen positional stability.

Example Cell Staining Protocol Method Using Medium Strength Hematoxylin Such as, for Example, Gill's #2 Hematoxylin.

Cells are typically prepared in ethanol and are purified or cultured using standard procedures prior to the following steps:
1. centrifuging a specimen for 5 minutes, aspirating off supernate and discarding supernate while retaining the resulting cell pellet;
2. resuspending the cell pellet in 50% ethanol, agitating well, centrifuging 5 minutes, aspirating and discarding supernate;
3. resuspending the cell pellet in tap water, agitating well, spinning for 5 minutes, aspirating, and discarding supernate;
4. repeating step 3;
5. resuspending the cell pellet in 1-1.5 ml of Gill Hematoxylin, agitating and allowing to sit 1 minute;
6. agitating well, spinning for 5 minutes, aspirating supernate and discarding;
7. resuspending the cell pellet in 3-5 ml tap water, agitating, spinning for 5 minutes, and discarding supernate;
8. repeating set 7;
9. resuspending the cell pellet in 3-5 ml tap water with 2-3 drops of ammonia, agitating, spinning for 5 minutes min, and discarding;
10. washing again in tap water, agitating, spinning and discarding supernate;
11. resuspending the cell pellet in 50% ethanol, agitating, spinning for 5 minutes, and discarding supernate;
12. resuspending the cell pellet in 80% ethanol, agitating, spinning for 5 minutes, and discarding supernate;
13. resuspending the cell pellet in 95% ethanol, agitating, spinning for 5 minutes, and discarding supernate;
14. repeating set 13 twice to extract as much cell water as possible;
15. resuspending the cell pellet in 100% ethanol, agitating, spinning, and discarding supernate;
16. repeating set 15 twice to assure dehydration;
17. transfering from poly centrifuging to glass tube after aspirating the final 100% wash supernate;
18. resuspending the cell pellet in 50/50 mixture of ethanol and xylene, then agitating, spinning and discarding supernate and repeating this step;
19. resuspending the cell pellet in pure xylene, agitating, spinning and discarding supernate. Repeating step 19 twice; and
20. resuspending the stained cell pellet in 1-2 ml of xylene, and storing at room temperature capped for future use.
   Example Method for Centrifugation of Cells into an Optical Gel Medium

The process of centrifugation of cells into an optical gel medium is as follows.
1. A small pool of gel is placed on a clean glass slide, and topped with a drop of xylene/cell slurry. A cover glass is placed onto the slide and gently compressed without mixing. Clarity is checked, as for example, under 100× oil magnification. Remaining water is rinsed out, as are ethanol traces that turn the gel cloudy. If the sample is cloudy, it is not acceptable for use. Cloudiness may sometimes be removed by further rinses.
2. 0.1 ml of gel is placed in a glass bottle. The bottle is capped and spun for 5 minutes at a setting that layers the gel onto the flat bottom of the tube.
3. The xylene/cell slurry of 0.3-0.6 ml is transferred onto the surface of the gel, and spun at the previous setting for 10 minutes. The supernate is thoroughly decanted and drained.
4. The remaining xylene is evaporated from the gel, returning the Nye OC431A optical gel, such as Nye OC431A optical gel, to its original viscosity.
   Actuation of Cells-in-Gel Medium

Once the cells are embedded in the high viscosity gel (herein called “cells-in-gel”), high pressure such as, in one example, greater than 1000 psi, using air, preferably with water vapor removed, or using mechanical pressure by applying a syringe plunger, will actuate the cells-in-gel through a microcapillary tube. Some useful microcapillary tubes have inner diameters of about 40-50 microns.

Imaging of Cells

Cells-in-gel are actuated through the microcapillary tube until a single cell appears in the field of view of the imaging system. Pressure is removed, and thus flow is stopped. The cylindrical shape of the cell medium in the microcapillary tube (or cells embedded in polymer threads, also cylindrically-shaped) allows access around 360 degrees normal to the cylinder axis; 180-degree access is critical for tomographic 3D imaging. For any view of the cell within the cylindrically shaped container, the carrier medium's refractive index is well matched throughout a volume between two flat parallel windows. This feature allows rotation and access for imaging through 360 degrees of rotation, but without significant optical distortion. Index matching using, for example, the average over visible wavelengths, between the Nye OC431A optical gel and the surrounding structures is within about 0.02 and produces a nearly-distortion free image as if there were no cylinder present. Only a few microns of the image on the inside of the microcapillary tube remain distorted.

Example Method for Cell Preparation for Buccal Scrapes in 3-D Visualization

General Sample Collection

An alternate embodiment of the method of the invention for buccal scrapes is described hereinbelow. Scrapings of the internal aspects of the oral cavity, that is, buccal surfaces of the cheek, are obtained as by using a plastic scraper or the like. Care should be taken to avoid abrading so vigorously as to cause bleeding. After scraping both left and right buccal surfaces, the scraper is placed into a container of isotonic solution for preservation of cytology specimens and for the liquefication of mucus. Mucoliquefying transport fluid for the collection and transport of fresh cytological specimens such as Mucolexx® available from Thermo Electric Corp., Pittsburgh, Pa., US, is used to cover the area containing the scrapings. The scraper is agitated very briskly for 20-30 seconds to dislodge any cellular material, then the scraper is removed and discarded.

The following steps are then carried out:
1. securely capping the specimen container immediately after the scraper is removed;
2. vigorously shaking the sample is for about 30 seconds manually or by using an automatic shaker in order to initiate maximizing mucolytic action in the sample;
3. allowing the specimen to settle for about 30 minutes;
4. aspirating the contents of the specimen container including Mucolexx and cellular material into the barrel of an empty syringe (note: no needle should be attached to the syringe), the syringe having sufficient volume to hold the entire contents;
5. quickly expelling the contents into a sample jar, and immediately re-aspirating the contents into the syringe, and continuing this motion for about 20-30 seconds to allow shearing forces to dislodge coincidental cell aggregates; and
6. returning the specimen to the collection jar and capping tightly.

Once the sample is shaken and syringed, it may be stored at room temperature for up to a week or more. If additional buccal samples from the same patient are being collected, they may be added to this container, followed by the required shaking period, and the combined sample may be kept at room temperature without cell deterioration.

Sample Concentration:

A method for increasing the sample concentration is carried out using the following steps:
1. shaking the specimen to thoroughly mix any cells that have sedimented to the bottom of the container including removing large sheets of cells and/or debris, by pouring the Mucolexx suspended cellular sample through a small pore-size kitchen sieve, discarding any trapped residue in the sieve and collecting the filtered cell suspension;
2. centrifuging the Mucolexx cell suspension at approximately at least 600 rpm for 5-7 minutes;
3. pipetting off the Mucolexx supernatant fluid, taking care not to dislodge any of the cell pellet in the bottom of the tube; and
4. if planning to store the sample for future use, resuspending in enough Mucolexx to at least triple the approximate volume of the centrifuged cell pellet. Labeled and capped plastic centrifuge cups may be used for storage since no xylene is involved.

A sample staining procedure using Hematoxylin is carried out using the following steps:
1. resuspending the centrifuged cell pellet in either distilled or tap water until the centrifuge cup is approximately half full and shaking to disperse the cellular elements;
2. centrifuging the sample at full speed for 5 minutes;
3. pipetting off the supernate and discarding without disturbing the cell pellet;
4. adding Hematoxylin to approximately double the cell pellet volume;
5. capping the tube and shaking the sample to distribute the cells in the dye and allowing settling for 1 minute;
6. centrifuging for 5 minutes, and then carefully pipetting off as much excess dye as possible without disturbing the pellet;
7. resuspending the pellet in water as by shaking, and centrifuging for 5 minutes, then pipetting off supernate and discarding the supernate;
8. repeating water rinse as noted above and pipetting off excess water;
9. adding dilute ammonia water in an amount of, for example, 2 drops pure ammonia per 3 ml tap water, to sample and shaking, then centrifuging as above and pipetting off supernate;
10. adding tap water and centrifuging as above, then pipetting off supernate;
11. adding and rinsing as above in 50% ethanol and pipetting off supernate;
12. rinsing in 80% ethanol, and pipetting off supernate;
13. rinsing in 95% ethanol, and pipetting off supernate;
14. rinsing step 13 at least twice more in 95% ethanol, pipetting and discarding supernate;
15. rinsing in 100% ethanol and pipetting and discarding supernate;
16. repeating rinsing in 100% ethanol at least twice more to remove any residual moisture trapped in the cellular elements to avoid cloudy preparations;
17. resuspending the residual cell pellet in xylene and place cell/xylene suspension in glass centrifuge tube, centrifuging specimen as above, and discarding supernate into toxic waste container;
18. repeating xylene rinse two more times, discarding the supernate appropriately in order to substantially remove all ethanol;
19. resuspending the cell pellet in xylene and shaking to disperse the cellular material;
20. allowing cell suspension to settle for about 20-30 seconds, then carefully pipetting off the supernate carrying the isolated cells in suspension and placing it in a second glass centrifuge cup; and
21. saving both tubes for capillary tube loading, the denser pellet might be useful later, but the better samples will come from the supernatant.
   Specimens prepared according to steps 1-21 may be stored for extended periods without appreciable cell loss or damage.

Cell Insertion into an optical system, such as a micro-capillary tube, is carried out using the following steps:
1. placing about 0.1-0.2 ml optical gel in bottom of a glass bottle having a capacity of about 1.0-2.0 ml.;
2. capping the bottle, centrifuge at high speed for 6-8 minutes to layer the gel onto the bottom of the bottle;
3. gently agitating a centrifuge cup with supernate cell suspension from step 20 above and then allowing settling for 15-20 seconds;
4. with non-corroding TB type syringe with a 27-gauge needle attached, carefully aspirating about 0.1-0.15 ml of cell suspension from approximately the middle third layer of the supernatant that has not settled to the bottom of the tube;
5. clearing off any cell clumps that might have been drawn into the tip of the needle that could clog the capillary tube, as by touching the needle tip gently and quickly to a paper towel;
6. gently expelling the cell/xylene sample onto the surface of the optical gel in the glass bottle;
7. capping the bottle and placing it in a centrifuge, spinning at high speed for 10-12 minutes;
8. when centrifugation is complete, uncapping and inverting the bottle on a paper towel to allow the xylene to drain off;
9. allowing the bottle to sit upright without a cap until ready for cell insertion, preferably in an exhaust hood, in order to let any remaining xylene evaporate;
10. with a micro-spatula, such as a small flat bladed screw driver scooping out the cell-laden portion of the gel, and inserting onto the inside wall of the barrel of the syringe portion of the capillary tube system;
11. adding a small portion of additional gel, and inserting the syringe plunger, gently pushing the gel/cell mass up to the tip of the syringe barrel;
12. placing the gel/cell-filled syringe in the coupling mechanism of the system, and, when substantially centered and stabilized, apply delicate pressure to the plunger, so as to expel gel into the chamber of the capillary tube; and
13. passing cells-in-gel through the capillary tube, and controlling or stopping the flow by applying positive or negative pressure to the plunger.
   Using Hydrodynamic Focusing for Centering Cells in Cylindrically-Shaped Medium

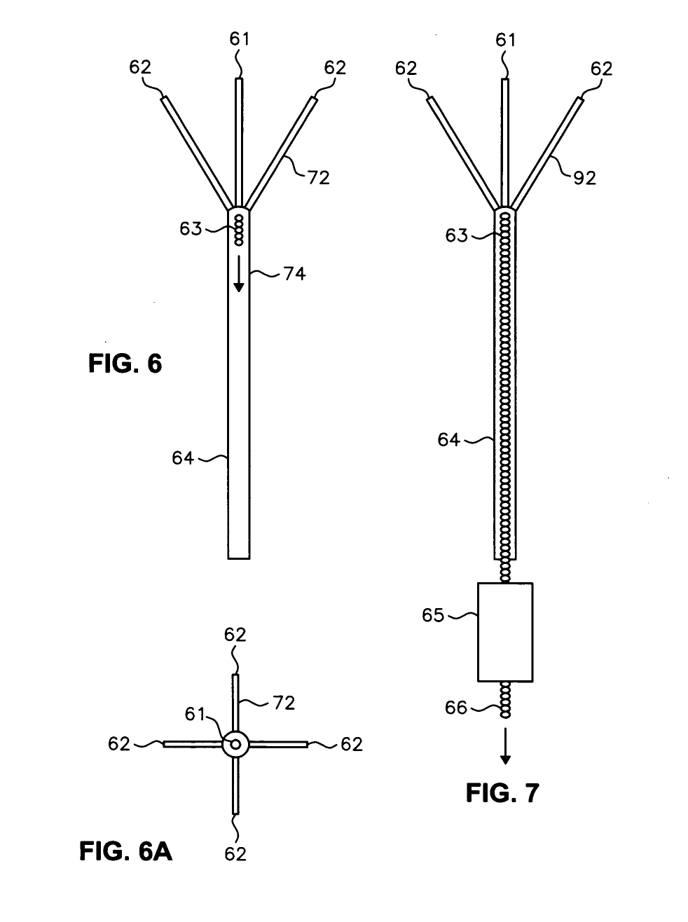

Referring now jointly to FIG. 6 and FIG. 6A, there schematically illustrated is a front view and end view respectively of a system for using hydrodynamic focusing for centering cells in cylindrically-shaped medium. After concentration of cells in the desired medium using the centrifugation methods described hereinabove, a high concentration (e.g. approximately 50% cells by volume) of a cell-medium mixture 61 is injected into a flow tube 64. A second medium 62 is injected into four or more ports 72. The second medium 62 advantageously comprises a medium without cells. At least two pairs of opposing flow streams of the second medium 62 serve to focus and center the cell-medium mixture 61 along two orthogonal axes, resulting in cells 63 centered within the microcapillary flow tube 64. Ideally, laminar flow without rippling is achieved for hydrodynamic focusing (Reynolds number Re<4 to 25 [See Transport Phenomena by Bird, Stewart, Lightfoot. John Wiley & Sons 1960]) in accordance with the relationship,

$$Re = \frac{ρ 〈 v 〉 D}{μ} ,$$

where ρ is density, <v> is average (characteristic) flow velocity, D is characteristic length and μ is (absolute) viscosity. In the case of a circular cross-section tube, the characteristic length D is the inner diameter of the microcapillary flow tube 64.

In order to embed cells in any medium, the cells are concentrated in the medium using centrifugation, with the average density of the cells nearly equal to that of the medium. This is necessary so that the cells are neutrally buoyant in the carrier medium. The cells quickly sediment out of the solvent, however, they must not sediment through the medium quickly, or the concentration of cells may not be increased. The rate of sedimentation of cells through the solvent must be much higher than the rate of sedimentation of cells through the medium in order to achieve increased cell concentration.

Referring now to FIG. 7, a side view of the system for using hydrodynamic focusing for centering cells in cylindrically-shaped medium as shown in FIG. 6 is schematically illustrated. Once the cell concentration has been increased, the cell-medium mixture 61 is injected substantially simultaneously with the four or more flow streams of medium 62 at a constant rate. When employing this methodology for embedding of cells within a polymer medium, it is preferable to use an ultra violet (UV) curing medium. Alternatively, other heat treatable polymer mediums or equivalent mediums suitable for cell embedding may be used. As the flow stream exits the microcapillary flow tube 64, a heating/curing assembly 65, such as, for example, a UV ring illuminator or heating mechanism, applies heat or UV light to the medium, as the case may be, hardening it. The flow stream is oriented vertically, pointing downward to avoid gravitational force applied laterally to the exiting flow stream. After passing through the heating/curing assembly 65 a linear polymer medium 66 is produced. As the linear polymer medium 66 cures during its fall downward, it can be wound up on a reel for storage. The linear polymer medium 66 may sometimes be characterized as a hardened cell thread.

If a non-curing media such as optical gel (e.g. Nye OC-431A or OC-431A-LVP), is used in place of a polymer as described above, a resultant cell-media mixture does not exit the tube and is not subject to a heating/curing assembly 65. The cell-gel mixture is instead actuated through the microcapillary tube 64 for viewing in an optical tomography system or other imaging system. The centration of the cells within the tube helps to retain contrast in pseudoprojection because it enables the range of objective scanning to be reduced. Improved centration also allows the total number of acquired projections to be reduced while still retaining the same resolution in a tomographically reconstructed 3D image.

In the case of 3D imaging of cells in a flow cytometer, a number of additional difficulties occur. Many images are acquired in series, and the registration of these images must be more accurate than the desired resolution of the system. For a 3D image to have a 0.5 micron resolution, the registration must be better than 0.5 micron (a 25% error is acceptable, that is, about 0.125 micron). This means that the rotational and translational motion of the cell must be very small, barring that motion along the flow axis. Using higher viscosity media with a flow system can reduce translational and rotational errors to an acceptable level, especially with symmetrically shaped cells that experience no stabilizing force that might prevent rotation. However, use of higher viscosity media necessitates a few changes from that used in standard flow cytometry. The focusing effect found with a single stream is due to the gradient of flow velocity, with an ideal laminar flow of an incompressible liquid yielding

$${v}_{z} = \frac{( {P}_{0} - {P}_{L} )}{4 µL} [ 1 - {( \frac{r}{R} )}^{2} ] .$$

Thus a parabolic velocity profile aids in focusing cells in a flow cytometer. However, as viscosity is increased, or if non-Newtonian fluids are used for transport, then the velocity gradient is reduced. Non-Newtonian fluids like a Bingham fluid may exhibit “plug flow” where the velocity profile is flat, having no gradient within a central region. When this occurs, hydrodynamic focusing using multiple input streams must be employed to achieve focusing, and hence centration of the cells.

The invention has been described herein in considerable detail in order to comply with the Patent Statutes and to provide those skilled in the art with the information needed to apply the novel principles of the present invention, and to construct and use such exemplary and specialized components as are required. However, it is to be understood that the invention may be carried out by specifically different equipment, devices and algorithms, and that various modifications, both as to the equipment details and operating procedures, may be accomplished without departing from the true spirit and scope of the present invention.

1. A method for embedding particles in a solid structure, the method comprising the steps of:
extruding a slurry of particles and a polymeric solution into a linear polymer medium having particles embedded into a polymer portion; and
curing the polymer portion of the linear polymer medium.

2. The method of claim 1, further comprising the step of inserting at least one micro-barcode into the slurry, such that the at least one micro-barcode is included in a segment of the linear polymer medium.

3. The method of claim 1, wherein the polymeric solution comprises a polymer, that, when cured, has an index of refraction matched with the index of refraction of a portion of the particles.

4. The method of claim 1, wherein the slurry is contained in a disposable container.

5. The method of claim 1, further comprising the step of using an injection device to regulate spacing between each specimen particle along the length of the linear polymer medium.

6. The method of claim 1, wherein the polymeric solution comprises a polymer substantially transparent to visible light.

7. The method of claim 1, wherein the particles comprise a biological specimen.

8. The method of claim 7 wherein the biological specimen comprises at least one of a cell, a human cell, a cancer cell, a cell nucleus and a microprobe.

9. A method for embedding particles in a solid structure, the method comprising the steps of:
micromolding a slurry including particles and a polymeric solution; and
curing the polymer portion of the slurry to form a solid specimen carrier.

10. The method of claim 9, further comprising the step of inserting at least one micro-barcode into the slurry, such that the at least one micro-barcode is included in a segment of the solid specimen carrier.

11. The method of claim 9, wherein the polymeric solution comprises a polymer, that, when cured, has an index of refraction matched with the index of refraction of a portion of the particles.

12. The method of claim 9, wherein the step of micromolding includes using a disposable mold.

13. The method of claim 9; comprising the intermediate step of including an injection device, said injection device serving to regulate the spacing between each object along the length of solid specimen carrier.

14. The method of claim 9, wherein the polymeric solution comprises a polymer substantially transparent to visible light.

15. The method of claim 9, wherein the particles comprise a biological specimen.

16. The method of claim 15 wherein the biological specimen comprises at least one of a cell, a human cell, a cancer cell, a cell nucleus and a microprobe.

17. A method for embedding particles in a solid structure, the method comprising the steps of:
pressurizing a slurry including particles and a polymeric solution to force the slurry into a microcapillary tube;
curing the polymer portion of the slurry to form a solid specimen carrier.

18. The method of claim 17, further comprising the step of inserting at least one micro-barcode into the slurry, such that the at least one micro-barcode is included in a segment of the solid specimen carrier.

19. The method of claim 17, wherein the polymer is selected to provide, upon solidification (curing), a matching of its index of refraction with the index of refraction of a portion of the particles contained in the slurry.

20. The method of claim 17, wherein the slurry is contained in a disposable container.

21. The method of claim 17, comprising the intermediate step of including an injection device, said injection device serving to regulate the spacing between each object along the length of the solid specimen carrier.

22. The method of claim 17, wherein the polymeric solution comprises a polymer substantially transparent to visible light.

23. The method of claim 17, wherein the particles comprise a biological specimen.

24. The method of claim 23 wherein the biological specimen comprises at least one of a cell, a human cell, a cancer cell, a cell nucleus and a microprobe.

25. A method for embedding particles in a solid structure, the method comprising the steps of:
pressurizing a slurry including particles and a polymeric solution to force the slurry into a microcapillary tube; and
vibrating the microcapillary tube to produce individual microspheres of hardened polymer.

26. The method of claim 25, wherein the polymeric solution is selected to provide, upon curing, a matching of its index of refraction with the index of refraction of a portion of the particles contained in the slurry.

27. The method of claim 25, wherein the slurry is contained in a disposable container.

28. The method of claim 25, wherein the polymeric solution comprises a polymer substantially transparent to visible light.

29. The method of claim 25, wherein the particles comprise a biological specimen.

30. The method of claim 31 wherein the biological specimen comprises at least one of a cell, a human cell, a cancer cell, a cell nucleus and a microprobe.

31. A method for using hydrodynamic focusing for centering cells in cylindrically-shaped medium comprising the steps of:
concentrating cells in a cell-medium mixture; and
injecting the cell-medium mixture into a microcapillary flow tube with a second medium injected using at least two pairs of opposing flow streams of the second medium that serve to focus and center the cell-medium mixture along two orthogonal axes, resulting in cells centered within the microcapillary flow tube.

32. The method of claim 31 wherein the step of injecting achieves laminar flow.

33. The method of claim 31 wherein the step of concentrating the cells comprises the step of concentrating cells in a polymer medium using centrifugation.

34. The method of claim 31 wherein the average density of cells in the cell-medium mixture is nearly equal to that of the medium.

35. The method of claim 31 wherein the second medium comprises a polymer medium.

36. The method of claim 31 wherein the second medium comprises an ultra violet curing medium.

37. The method of claim 31 wherein the second medium comprises a heat treatable polymer medium.

38. The method of claim 37 further comprising the step of applying radiation to a flow stream exiting the microcapillary flow tube.

39. The method of claim 38 wherein the flow stream is oriented vertically.

40. A method for step flow actuation of cells in an imaging system including a field of view and a microcapillary tube, the method comprising the steps of:
transferring cells into a solvent,
embedding the resulting cell/solvent mixture in a carrier medium having a viscosity greater than 10 centipoises;
applying pressure to actuate the cells embedded in gel through the microcapillary tube until a single cell appears in the field of view of the imaging system; and
removing pressure to stop flow.

41. The method of claim 40 wherein the carrier medium viscosity is greater than 100 centipoises.

42. The method of claim 40 wherein the carrier medium viscosity is greater than 1,000 centipoises.

43. The method of claim 40 wherein the carrier medium viscosity is greater than 1 million centipoises.

44. The method of claim 40 wherein the step of applying pressure includes applying pressure greater than 1000 psi.

45. The method of claim 40 wherein the solvent comprises xylene.

46. The method of claim 40 wherein the step of embedding comprises centrifugation of the resulting cell/solvent mixture into an optical gel.

47. The method of claim 40 wherein the cells comprise cells from buccal scrapes.
